(19) United States
(12) Patent Application Publication
(10) Pub. No.: US 2023/0389842 A1
(43) Pub. Date: Dec. 7, 2023

(54) INFERRING PSYCHOLOGICAL STATE

(71) Applicant: Hewlett-Packard Development Company, L.P., Spring, TX (US)

(72) Inventors: Erika SIEGEL, Palo Alto (CA); Rafael Ballagas, Palo Alto, CA (US); Srikanth Kuthuru, Palo Alto, CA (US); Jishang Wei, Guilford, CT (US); Hiroshi Horii, Palo Alto, CA (US); Alexandre Santos Da Silva Jr, Porto Alegre (BR); Jose Dirceu Grundler Ramos, Porto Alegre (BR); Rafael Dal Zotto, Spring, TX (US); Gabriel Lando, Porto Alegre (BR)

(21) Appl. No.: 18/247,776

(22) PCT Filed: Oct. 5, 2020
(86) PCT No.: PCT/US2020/054259

§ 371 Date: Apr. 4, 2023

(51) Int. Cl. A61B 5/16; A61B 5/11; G16H 50/20

(52) U.S. Cl. CPC A61B 5/165; A61B 5/1116; G16H 50/20

(57) ABSTRACT

Methods, systems, apparatus, and computer-readable media (transitory or non-transitory) are described herein for inferring psychological states. In various examples, data indicative of a measured affect of an individual may be processed using a regression model to determine a coordinate in a continuous space. The continuous space may be indexed based on a plurality of discrete psychological labels. In a first context, the coordinate in the continuous space may be mapped to one of a first set of the discrete psychological labels associated with the first context. In a second context, the coordinate in the continuous space may be mapped to one of a second set of the discrete psychological labels associated with the second context.

### BACKGROUND

An individual's affect is a set of observable manifestations of an emotion or cognitive state experienced by the individual. An individual's affect can be sensed by others, who may have learned, e.g., through lifetimes of human interactions, to infer an emotional or cognitive state (either constituting a “psychological state”) of the individual. Put another way, individuals are able to convey their emotional and/or cognitive state through various different verbal and non-verbal cues, such as facial expressions, voice characteristics (e.g., pitch, intonation, and/or cadence), and bodily posture, to name a few.

### BRIEF DESCRIPTION OF THE DRAWINGS

Features of the present disclosure are illustrated by way of example and not limited in the following figure(s), in which like numerals indicate like elements.

FIG. 1 schematically depicts an example environment in which selected aspects of the present disclosure may be implemented.

FIGS. 2A, 2B, and 2C demonstrate an example of how different affectual datasets may be mapped to the same continuous space, in accordance with various examples.

FIG. 3 depicts an example Voronoi plot that may be used to map continuous space coordinates to regions corresponding to discrete psychological labels, in accordance with various examples.

FIG. 4 schematically depicts an example architecture for preprocessing data in accordance with aspects of the disclosure.

FIG. 5 depicts an example method for mapping an affectual dataset to a continuous space, including training a model, in accordance with various examples.

FIG. 6 depicts an example method for inferring psychological states, in accordance with various examples.

FIG. 7 shows a schematic representation of a system, according to an example of the present disclosure.

FIG. 8 shows a schematic representation of a non-transitory computer-readable medium, according to an example of the present disclosure.

### DETAILED DESCRIPTION

An individual's facial expression may be captured using sensor(s), such as a vision sensor, and analyzed by a data processing device, such as a computer to infer the individual's psychological state. However, existing techniques are limited to predicting a narrow set of discrete psychological states. Moreover, different cultures may tend to experience and/or exhibit psychological states differently. Consequently, discrete psychological states associated with one culture may not be precisely aligned with those of another culture.

Another challenge is access to affectual data that is suitable to train model(s), such as regression models, to infer psychological states. Publicly-available affectual datasets related to emotion and cognition are often too small, too specific, and/or are labeled in a way that is incompatible with a particular goal. Moreover, unsupervised clustering of incongruent affectual datasets in the same continuous space may be ineffective since there is no guarantee that two clusters of data that have semantically-similar labels will be proximate to each other in the continuous space. While it is possible for a data science team to collect its own affectual data, internal data collection is expensive and time consuming.

Examples are described herein for jointly mapping incongruent affectual datasets into the same continuous space to facilitate context-specific inferences of individuals' psychological states. In some examples, each affectual dataset may include instances of affectual data (e.g., sensor data capturing aspects of individuals' affects) and a set or “palette” of psychological labels used to describe (or “label”) each instance of affectual data. As will be discussed in more detail, the palette of psychological labels associated with each affectual dataset may be applicable in some context(s), and less applicable in others. Put another way, a palette of psychological labels associated with an affectual dataset may include emotions and/or cognitive states that are expected to be observed under a context/circumstance with which the affectual dataset is aligned, compatible, and/or semantically relevant.

In various examples, data indicative of a measured affect of an individual may be captured, e.g., using sensors such as vision sensors (e.g., a camera integral with or connected to a computer), microphones, etc. This data may be processed using a model such as a regression and/or machine learning model to determine a coordinate in a continuous space. The continuous space may have been previously indexed based on a plurality of discrete psychological labels. Accordingly, the coordinate in the continuous space may be used to identify the closest of the discrete psychological labels, e.g., using a Voronoi plot that partitions the continuous space into regions close to each of the discrete psychological labels.

In some examples, output indicative of the closest discrete psychological label may be rendered at a computing device, e.g., to convey the individual's inferred psychological state to others. For instance, in a video conference with multiple participants, one participant may be presented with inferred psychological states of other participant(s). As another example, a presenter may be provided with (e.g., at a display in front of them) inferred psychological states of audience members, aiding the presenter in “reading the room.”

In some examples, the continuous space is multi-dimensional and includes multiple axes. In some examples, the continuous space is two-dimensional, with one axis corresponding to valence and another axis corresponding to arousal. In other examples, a two-dimensional continuous space may include a hedonic axis and an activation axis. These axes may be used as guidance for mapping a plurality of discrete psychological states available in incongruent affectual datasets to the same continuous space.

For example, a user may map each discrete psychological label (e.g., happy, sad, angry) available in a first affectual dataset along these axes based on the user's knowledge and/or expertise. Additionally, the same user or a different user may map each discrete psychological label (e.g., bored, inattentive, disgusted, distracted) available in a second affectual dataset that is incongruent with the first affectual dataset along the same axes based on the user's knowledge and/or expertise.

Once the continuous space is indexed based on these discrete psychological labels, a model, such as the aforementioned regression and/or machine learning model, may be trained to map the affectual data to coordinates in the continuous space that correspond to the discrete psychological labels of the affectual datasets. After training and during inference, subsequent unlabeled affectual data may be processed using the trained model in order to generate coordinates in the continuous space, which in turn can be used to identify discrete psychological labels as described above.

In some examples, an advantage of mapping multiple incongruent affectual datasets into a single continuous space (and training a predictive model accordingly) is that it is possible to dynamically make inferences that are specific to particular semantic contexts/circumstances. For example, an English-speaking video conference participant may wish to see psychological inferences in English, whereas a Korean-speaking video conference participant may wish to see psychological inferences in Korean. Assuming both English and Korean affectual datasets have already been mapped to the same continuous space (and the model has been adequately trained), the English-speaking video conference participant may receive output that conveys psychological inferences in English, whereas the Korean-speaking video conference participant may receive output that conveys psychological inferences in Korean.

Examples described herein are not limited to linguistic translation between psychological states in different languages. As noted previously, different cultures may tend to experience and/or exhibit psychological states differently. As another example, a business video conference may warrant inference from a different palette of psychological labels/states than, for instance, a social gathering such as a film “watch party” with others over a network. As yet another example, a virtual travel experience may warrant inference from a different “palette” of psychological labels than a first-person shooter gaming experience. Additionally, different roles of individuals can also evoke different contexts. For example, a teacher may find utility in inferences drawn from a different palette of emotions than a student.

Accordingly, context-triggered transitions between incongruent sets of psychological states may involve semantic adaptation, in addition to or instead of linguistic translation. And this semantic adaptation may be based on various contextual signals associated with a first individual to which inferred psychological states are presented and/or with a second individual from which psychological states are inferred. These contextual signals may include, but are not limited to, an individual's location, role/title, current activity, relationship with others, demographic(s), nationality, user preferences, membership in a group (e.g., employment at a company), vital signs, and observed habits, to name a few.

For example, an affectual dataset that includes a palette of psychological labels associated with a dining context, such as “ravenous,” “repulsed,” “thirsty,” “indifferent,” and “satisfied,” may be less applicable in a different semantic context, such a film test audience. However, if this palette of psychological labels is jointly mapped to the same continuous space as another palette of psychological labels associated with another, more contextually-suitable affectual dataset (e.g., a dataset associated with attention/enjoyment), as described herein, then it is possible to semantically transition between the incongruent sets of psychological labels, allowing for psychological inferences from either.

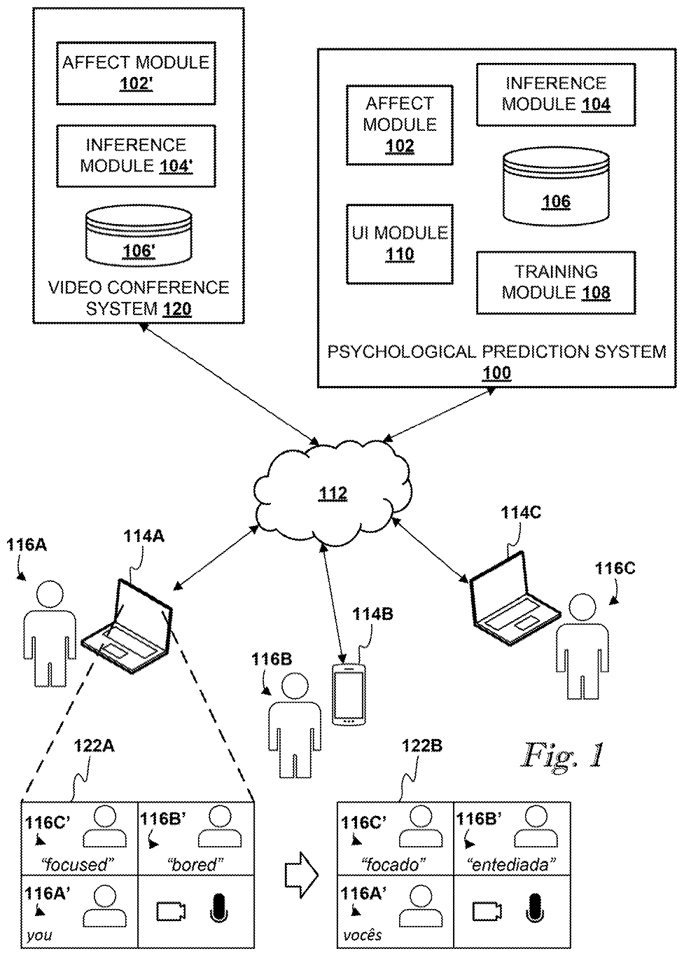

FIG. 1 schematically depicts an example environment in which selected aspects of the present disclosure may be implemented. A psychological prediction system 100 may include various components that, alone or in combination, perform selected aspects of the present disclosure to facilitate inference of psychological states. Each of these components may be implemented using any combination of hardware and computer-readable instructions. In some examples, psychological prediction system 100 may be implemented across computing systems that collectively may be referred to as the “cloud.”

An affect module 102 may obtain and/or receive biometric data and/or other affectual data indicative of an individual's affect from a variety of different sources. As noted previously, an individual's affect is a set of observable manifestations of an emotion or cognitive state experienced by the individual. Individuals are able to convey their emotional and/or cognitive state through various different verbal and non-verbal cues, such as facial expressions, voice characteristics (e.g., pitch, intonation, and/or cadence), and bodily posture, to name a few. These cues may be detected using various types of sensors, such as microphones, vision sensors (e.g., 2D RGB digital cameras integral with or connected to personal computing devices), infrared sensors, physiological sensors (e.g., to detect heartrate, blood oxygen levels, temperatures, sweat level, etc.), and so forth.

The affectual data obtained/received by affect module 102 may be processed, e.g., by an inference module 104, based on various regression and/or machine learning models that are stored in a model database 106. The output generated by inference module 104 based on these affectual data may include and/or be indicative of the individual's psychological state, which can be an emotional state and/or a cognitive state.

Psychological prediction system 100 also includes a training module 108 and a user interface (UI) module 110. Training module 108 may create, edit, and/or update (collectively, “train”) model(s) that are stored in model index 106 based on training data. Training data may include, for instance, labeled data for supervised learning, unlabeled data for unsupervised learning, and/or some combination thereof for semi-supervised learning. Additionally, training data may include affectual datasets that exist already or that can be created as needed. An affectual dataset may include a plurality of affectual data instances that is harvested from a plurality of individuals. Each affectual data instance may represent and/or be indicative of a set of observable manifestations of an emotion or cognitive state experienced by a respective individual.

In some examples, inference module 104 and training module 108 may cooperate to train model(s) in model index 106. For example, inference module 104 may process training example(s) based on a model from index 106 to generate output. Training module 108 may compare this output to label(s) associated with the training example(s). Any difference or “error” between the output and the label(s) may be used by training module 108 to train the model(s), e.g., using techniques like regressive analysis, gradient descent, back propagation, etc.

Various types of model(s) may be stored in index 106 and used, e.g., by inference module 104, to infer psychological states. Regressive models may be employed in some examples, and may include, for instance, linear regression models, logistic regression models, polynomial regression models, stepwise regression models, ridge regression models, lasso regression models, and/or ElasticNet regression models, to name a few. Other types of models may be employed in other examples. These other models may include, but are not limited to, support vector machines, Bayesian networks, decision trees, various types of neural networks (e.g., convolutional neural networks, feed-forward neural networks, various types of recurrent neural networks, transformer networks), random forests, and so forth. Regression models and machine learning models are not mutually exclusive. As will be described below, in some examples, a multi-layer perceptron (MLP) regression model may be used, and may take the form of a feed-forward neural network.

Psychological prediction system 100 may be in network communication with a variety of different data processing devices over computing network(s) 112. Computing network(s) 112 may include, for instance, a local area network (LAN) and/or a wide area network (WAN) such as the Internet. For example, in FIG. 1, psychological prediction system 100 is in network communication with three personal computing devices 114A-C operated, respectively, by three individuals 116A-C.

In this example, first personal computing device 114A and third personal computing device 114C take the form of laptop computers, and second personal computing device 114B takes the form of a smart phone. However, the types and form factors of computing devices that allow individuals (e.g., 116A-C) to take advantage of techniques described herein are not so limited. While not shown in FIG. 1, personal computing devices 114A-C may be equipped with various sensors (e.g., cameras, microphones, other biometric sensors) mentioned previously that can capture different types of affectual data from individuals 116A-C.

In the example of FIG. 1, individuals 116A-C are using their respective personal computing devices 114A-C to participate in a video conference. The video conference is facilitated by a video conference system 120. However, techniques described herein for inferring psychological states are not limited to video conferences, and the example of FIG. 1 is included simply for illustrative purposes. Psychological states inferred using techniques described herein may be applicable in a wide variety of applications. Some examples include allowing a speaker of a presentation or a moderator of a test audience to gauge the attentiveness and/or interest of audience members. Psychiatrists and/or psychologists may use inferences generated using techniques described herein to infer psychological states of their patients. Social workers and other similar personnel may leverage techniques described herein to, for instance, perform wellness checks.

Individuals 116A-C may communicate with each other as part of a video conference facilitated by video conference system 120 (and in this context may be referred to as “participants”). Accordingly, each individual 116 may see graphical representations of other individuals (participants) participating in the video conference, such as avatars and/or live streams. An example of this is shown in the called-out window 122A at bottom left, which demonstrates what first individual 116A might see while participating in a video conference with individuals 116B and 116C. In particular, graphical representations 116C′ and 116B′ are presented in a top row, and first individual's own graphical representation 116A′ is presented at bottom left. Controls for toggling a camera and/or microphone on/off are shown at bottom right.

In this example, a psychological inference of “focused” is rendered under graphical representation 116C′ of third individual 116C. Inference module 104 of psychological prediction system 100 may have made this inference based on affectual data captured by, for instance, a webcam onboard third personal computing device 116C. A psychological inference of “bored” is rendered under graphical representation 116B′ of second individual 116B. Inference module 104 of psychological prediction system 100 may have made this inference based on affectual data captured by, for instance, a camera and/or microphone integral with second personal computing device 116B.

As noted above, at bottom left, individual 116A may see his or her own graphical representation. In this example, it is simply labeled as “you” to indicate to individual 116A that they are looking at themselves, or at their own avatar if applicable. However, in some examples, individuals can elect to see psychological inferences made for themselves, e.g., if they want to know how they appear to others during a video conference. For example, individual 116A may operate settings of his or her video conference client to toggle his or her own psychological state on or off. In some examples, individuals may have the option of preventing inferences made about them from being presented to other video conference participants, e.g., if they wish to maintain their privacy.

In some examples, the psychological inferences that are generated and presented to individuals, e.g., as part of a video conference, are context-dependent. For example, if individual 116A speaks English, they may desire to see psychological inferences about others in English, as presented in window 122A. However, if individual 116A were Brazilian, they may desire to see psychological inferences presented in Portuguese, as shown in the alternative window 122B.

This context may be selected by individual 116A manually and/or may be determined automatically. For example, individual 116A may have configured his or her personal computing device 114A (e.g., during setup) as being located in Brazil. Alternatively, a position coordinate sensor such as a Global Positioning system (GPS) sensor integral with or otherwise in communication with personal computing device 114A may indicate that individual 116A is located in Brazil. For example, a phone (not depicted) carried by individual 116A may include a GPS sensor that provides a current position to personal computing device 114A, e.g., via a personal area network implemented using technology such as Bluetooth.

Regardless of how the context (or circumstance) is determined, individual 116A may be presented with the content of window 122B, which includes Portuguese inferences. In window 122B, the psychological inference presented underneath graphical representation 116C′ of third individual 116C is “focado” instead of “focused.” Similarly, the psychological inference presented underneath graphical representation 116B′ of second individual 116B is “entediada” instead of “bored.” And instead of seeing “you” at bottom left, individual 116A may see “vocês.”

Psychological prediction system 100 does not necessarily process every psychological inference locally. In some examples, psychological prediction system 100 may, e.g., via training module 108, generate, update, and/or generally maintain various models in index 106. The models in index may then be made available to others, e.g., over network(s) 112.

For example, in FIG. 1, video conference system 120 includes its own local affect module 102′, local inference module 104′, and a local model index 106′. Local affect module 102′ may receive various affectual data from sensors integral with or otherwise in communication with personal computing devices 114A-C, similar to remote affect module 102 of psychological prediction system 100. Local inference module 104′ may, e.g., periodically and/or on demand, obtain updated models from psychological prediction system 100 and store them in local model index 106′. Local inference module 104′ may then use these models to process affectual data obtained by local affect module 102′ to make inferences about video conference participants' psychological states.

UI module 110 of psychological prediction system 100 may provide an interface that allows users (e.g., individuals 116A-C) to interact with psychological prediction system 100 for various purposes. In some examples, this interface may be an application programming interface (API). In other examples, UI module 110 may generate and publish markup language documents written in various markup languages, such as the hypertext markup language (HTML) and/or the extensible markup language (XML). These markup language documents may be rendered, e.g., by a web browser of a personal computing device (e.g., 116A-C), to facilitate interaction with psychological prediction system 100.

In some examples, users may interact with UI module 110 to create and/or onboard new affectual datasets with labels that can be the basis for new sets of psychological inferences. For example, a new affectual dataset that includes instances of affectual training data labeled with psychological (e.g., emotional and/or cognitive) labels may be provided to inference module 104. A user may interact with UI module 110 in order to map those new psychological states/labels associated with the new affectual dataset to a continuous space.

Once the labels are mapped to the continuous space, inference module 104 and training module 108 may cooperate to train model(s) in model index 106 to predict those labels based on the affectual dataset, thereby mapping the affectual dataset to those labels in the continuous space. Other affectual datasets with different labels may also be mapped to the same continuous space in a similar fashion. By mapping multiple incongruent affectual datasets to the same continuous space, it is possible to transition between different, incongruent sets of psychological labels, e.g., based on context. Thus, for instance, individual 116A is able to switch from seeing psychological inferences in English to seeing psychological inferences in Portuguese.

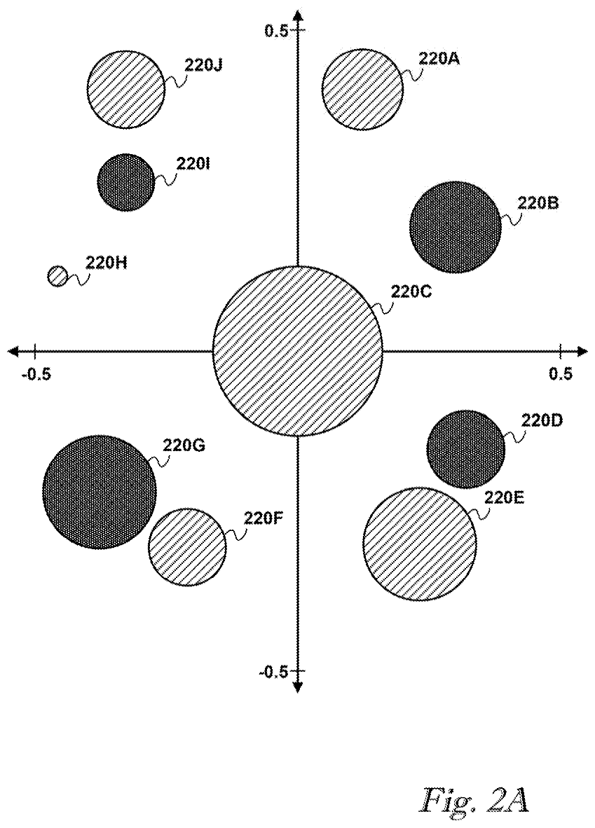

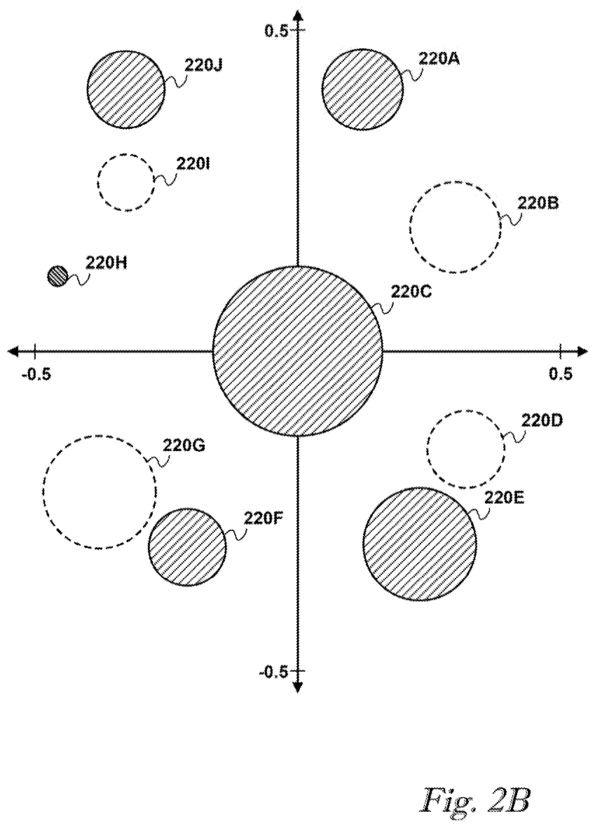

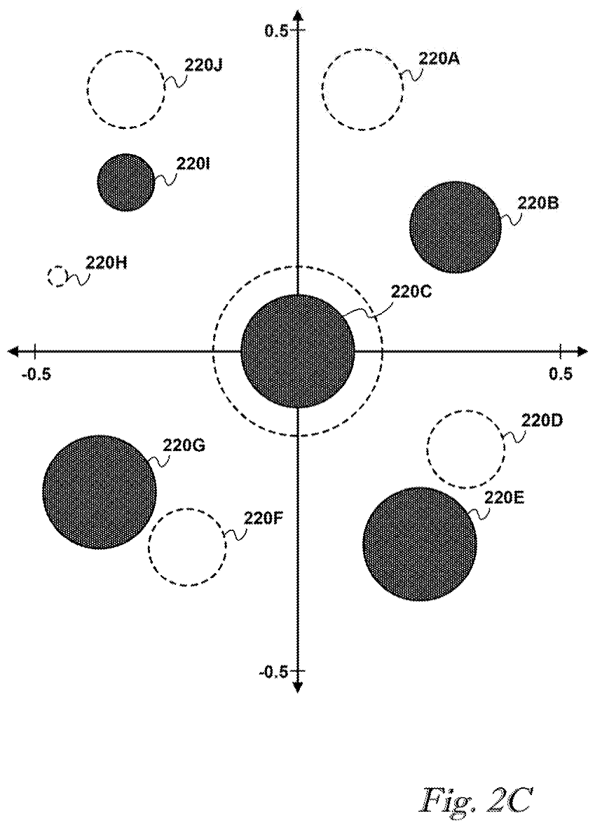

FIGS. 2A, 2B, and 2C demonstrate an example of how different affectual datasets may be mapped to the same continuous space, in accordance with various examples. In some examples, a GUI may present an interface that visually resembles FIGS. 2A-C, and that allows a user to manually map various psychological labels associated with various incongruent affectual datasets to the same continuous space.

As used herein, a first affectual dataset is incongruent with a second affectual dataset where, for instance, the psychological labels of the first affectual dataset are different than those of the second affectual dataset. In some cases, sets of labels associated with incongruent affectual datasets may be disjoint from each other, although this is not always the case. For example, one affectual dataset designed to capture one set of emotions may include the labels “happy,” “sad,” “excited,” and “bored.” Another affectual dataset designed to capture another set of emotions may include the labels “amused,” “anxious,” “disgusted,” and “scared.”

Referring to FIG. 2A, the interface depicts a two-dimensional continuous space with two axes. The horizontal (or X) axis may represent, for instance, valence, and includes a range from −0.5 to 0.5. The vertical (or Y) axis may represent, for instance, arousal, and also includes a range from −0.5 to 0.5. These axes and ranges are not limiting; in other examples, the axes may include a hedonic axis and an activation axis, for instance, and may utilize other ranges, such as [0, 1], [−1, 1], etc.

In FIG. 2A a user has manually positioned a plurality of discrete psychological labels 220A-J associated with affectual datasets onto the continuous space, e.g., based on the user's own experience and/or expertise. The circles have two different fill patterns (diagonal lines and dark fill) that correspond to two incongruent affectual datasets. Thus, psychological labels 220A, 220C, 220F, 220H, and 220J are associated with one affectual dataset. Psychological labels 220B, 220D, 220G, and 220I are associated with another affectual dataset.

These psychological labels are mapped by a user on the axes as shown. For example, first discrete psychological label 220A has a very positive arousal and a somewhat positive valence, and may correspond to, for instance, “surprise.” Second discrete psychological label 220B has a lower arousal value but a greater valence value, and may correspond to, for instance, “happy.”

Third discrete psychological label 220C is positioned around the center of both axes, and may represent “neutral,” for example. Fourth discrete psychological label 220D has a relatively large valence but a slightly negative arousal value, and may correspond to, for instance, “calm.” Fifth discrete psychological label 220E has a somewhat smaller valence but a slightly lower arousal value, and may correspond to a psychological state similar to calm, such as “relaxed.”

Sixth discrete psychological label 220F has a slightly negative valence and a more pronounced negative arousal value, and may correspond to, for instance, “bored.” Seventh discrete psychological label 220G has a more negative valence than 220F and a less pronounced negative arousal value, and may correspond to, for instance, “sad.”

Eighth discrete psychological label 220H has very negative valence and a somewhat positive arousal value, and may correspond to, for instance, “disgust.” Ninth discrete psychological label 220I has a less negative valence than 220H and a greater arousal value, and may correspond to, for instance, “anger.” Tenth discrete psychological label 220J has a similar negative valence as 220I and a greater arousal value, and may correspond to, for instance, “fear.”

In some examples, the user may place these discrete psychological labels 220A-J on the continuous space manually, e.g., using a pointing device to drag the graphical elements (circles) representing the psychological labels to desired locations. The user may also adjust other aspects of the discrete psychological labels 220A-J, such as their sizes and/or shapes. For example, while discrete psychological labels 220A-J are represented as circles, this is not meant to be limiting; they can have any shape desired by a user.

Additionally, and as shown, different discrete psychological labels 220A-J can have different sizes to represent, for instance, different probabilities or frequencies of those labels occurring amongst training examples in their corresponding affectual datasets. In some examples, the sizes/diameters of discrete psychological labels 220A-J may be adjustable, and may correspond to weights that are used to determine which psychological label is applicable in a particular inference attempt. For example, disgust (220H) may be encountered relatively infrequently in an affectual dataset, such that the user would prefer that sadness (220I) or fear (220J) be more easily/frequently inferred.

In some examples, various discrete psychological labels 220A-J may be activated or deactivated depending on the context and/or circumstances. An example of this was demonstrated previously in FIG. 1 with the English inferences presented in window 122A verses the Portuguese inferences presented in window 122B. FIGS. 2B and 2C provide another example.

In FIG. 2B, various discrete psychological labels, including 220B, 220D, 220G, and 220I have been deactivated, as indicated by the dashed lines and lack of fill. Accordingly, the remaining discrete psychological labels, 220A, 220C, 220E, 220F, 220H, and 220J are active. Thus, with the configuration shown in FIG. 2B, an inferences made by inference module 104 (or 104′) may be mapped to one of the remaining active discrete psychological labels.

In FIG. 2C, various discrete psychological labels, including 220A, 220D, 220F, 220H, and 220J have been deactivated, as indicated by the dashed lines and lack of fill. Accordingly, the remaining discrete psychological labels, 220B, 220E, 220G, and 220I are active. Thus, with the configuration shown in FIG. 2B, an inferences made by inference module 104 (or 104′) may be mapped to one of the remaining active discrete psychological labels. Discrete psychological label 220C remains active in FIG. 2C, but has a smaller diameter to indicate that it occurred less frequently in the underlying affectual training data, and/or should be detected less frequently, than the corresponding psychological state 220C in FIG. 2B.

When affectual data gathered, e.g., at a personal computing device 116, is processed by inference module 104 (or 104′), the output may be, for instance, a coordinate in continuous space. For example, in reference to the continuous space depicted in FIGS. 2A-C, the output may be a two-dimensional coordinate such as [0.25, 0.25], which would define a point in the top right quadrant. As shown in FIGS. 2A-C, there is no guarantee that such a coordinate will fall into one of the psychological states 220A-J.

In some examples, therefore, the nearest discrete psychological state 220 to a coordinate in continuous space output by inference module 104 may be identified using techniques such as the dot product and/or cosine similarity. In other examples, the coordinate in the continuous space may be mapped to one of a set of the discrete psychological labels is performed using a Voronoi plot that partitions the continuous space into regions close to each of the set of discrete psychological labels.

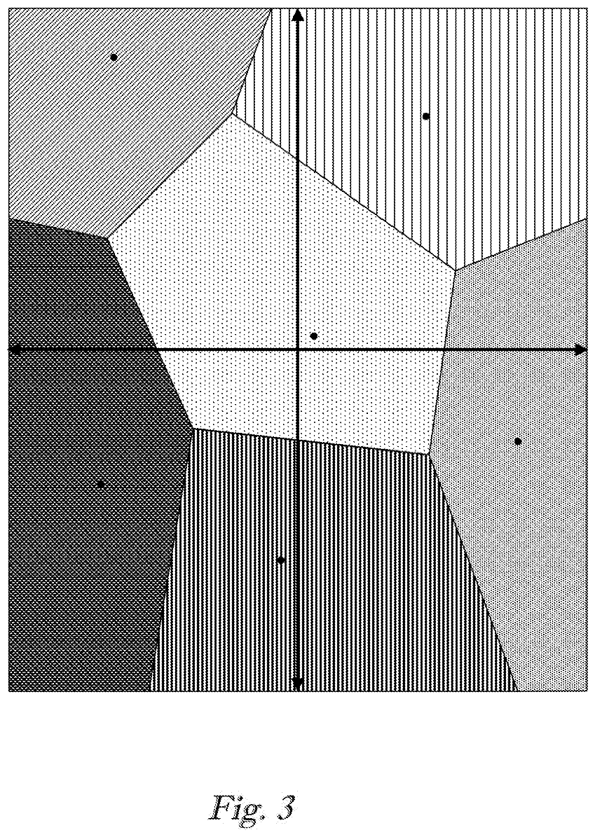

FIG. 3 depicts an example Voronoi plot that may be used to map continuous space coordinates to regions corresponding to discrete psychological labels, in accordance with various examples. In FIG. 3, multiple black dots called “seeds” are shown at various positions. Each seed correspond to a different discrete psychological label.

In FIG. 3, each seed is contained in a corresponding region that includes all points of the continuous space that are closer to that seed than to any other. These regions are called Voronoi “cells.” Upon new (e.g., unlabeled) affectual data being processed by inference module 104 to make an inference, the continuous space coordinates may be mapped onto a Voronoi plot like that shown in FIG. 3. Whichever region captures the coordinate also identifies the psychological state that is inferred.

In some examples, discrete psychological labels such as those depicted in FIGS. 2A-C may be used to generate a Voronoi plot similar to that depicted in FIG. 3. The Voronoi plot is in fact a visualization of applying a nearest neighbor technique towards locations outside of the circular regions depicted in 2A-C.

Data indicative of the affect of an individual—which as noted above may include sensor data that captures various characteristics of the individual's facial expression, body language, voice, etc.—may come in various forms and/or modalities. For example, one affectual dataset may include vision data acquired by a camera that captures an individual's facial expression and bodily posture. Another affectual dataset may include vision data acquired by a camera that captures an individual's bodily posture and characteristics of the individual's voice contained in audio data captured by a microphone. Another affectual dataset may include data acquired from sensors onboard an extended reality headset (augmented or virtual reality), or onboard wearables such as a wristwatch or smart jewelry.

In some examples, incongruent affectual datasets may be normalized into a form that is uniform, so that inference module 104 is able to process them using the same model(s) to make psychological inferences. For example, in some examples, multiple incongruent affectual datasets may be preprocessed to generate embeddings that are normalized or uniform (e.g., same dimension) across the incongruent datasets. These embeddings may then be processed by inference module 104 using model(s) stored in index 106 to infer psychological states.

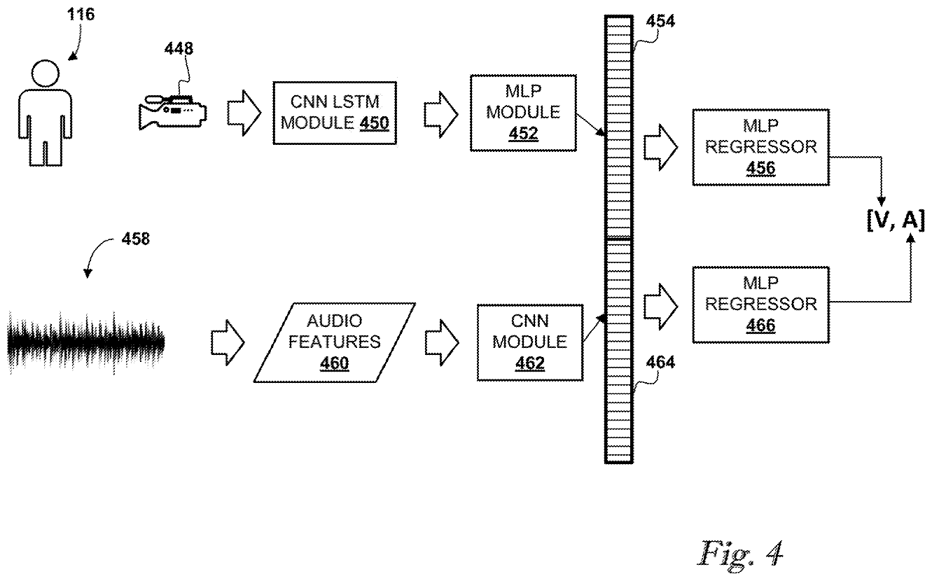

FIG. 4 schematically depicts an example architecture for preprocessing data in accordance with aspects of the disclosure. Various features of an affect of an individual 116 are captured by a camera 448. These features may be processed using a convolutional long short-term memory neural network (CNN LSTM) 450. Output of CNN LSTM 450 may be processed by a MLP module 452 to generate an image embedding 454.

Meanwhile, audio data 458 (e.g., a digital recording) of the individual's voice may be captured by a microphone (not depicted). Audio features 460 may be extracted from audio data 458 and processed using a CNN module 462 to generate an audio embedding 464. In some examples, visual embedding 454 and audio embedding 464 may be combined, e.g., concatenated, as a single, multi-modal embedding 454/464.

This single, multi-modal embedding 454/464 may then be processed by multiple MLP regressor models 456, 466, which may be stored in model index 106. As noted previously, regression models are not limited to MLP regressor models. Each MLP regressor model 456, 466 may generate a different numerical value, and these numerical values may collectively form a coordinate in continuous space. In FIG. 4, for instance, MLP regressor model 456 generates the valence value along the horizontal axis in FIGS. 2A-C. MLP regressor 466 generates the arousal value along the vertical axis in FIGS. 2A-C.

The architecture of FIG. 4 may be used to process multi-modal affectual data that includes both visual data captured by camera 448 and audio data 458. Other affectual datasets having different modalities may be processed using different architectures to generate embeddings that are similar to combined embedding 454/464, and/or that are compatible with MLP regressor models 456, 466.

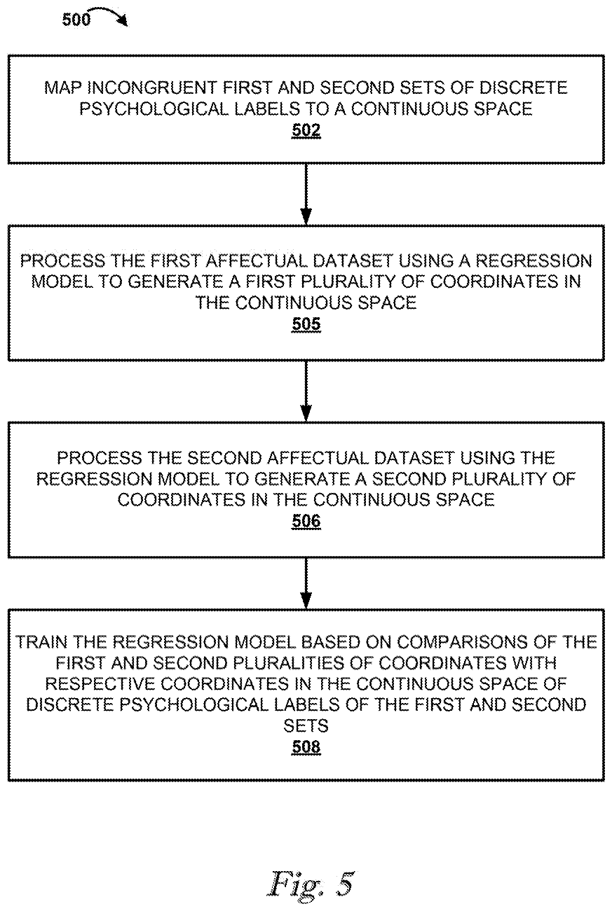

FIG. 5 depicts an example method 500 for mapping an affectual dataset to a continuous space, including training a model, in accordance with various examples. For convenience, the operations of method 500 will be described as being performed by a system, which may include, for instance, psychological prediction system 100. The operations of method 500 may be reordered, and various operations may be added and/or omitted.

At block 502, the system may map incongruent first and second sets of discrete psychological labels to a continuous space. The first set of discrete psychological labels may be used to label a first affectual dataset (e.g., facial expression plus voice characteristics). The second set of discrete psychological labels may be used to label a second affectual dataset (e.g., facial expression alone). For example, a user may operate a GUI that is rendered in cooperation with UI module 110 in order to position the incongruent first and second sets of discrete psychological labels into the two-dimensional space depicted in FIGS. 2A-C.

At block 504, the system, e.g., by way of inference module 104 and/or training module 108, may process the first affectual dataset using a regression model (e.g., MLP regressor model 456 and/or 466) to generate a first plurality of coordinates in the continuous space. At block 506, the system, e.g., by way of inference module 104 and/or training module 108, may process the second affectual dataset using the regression model (e.g., MLP regressor model 456 and/or 466) to generate a second plurality of coordinates in the continuous space.

At block 508, the system, e.g., by way of training module 108, may train the regression model (e.g., MLP regressor model 456 and/or 466) based on comparisons of the first and second pluralities of coordinates with respective coordinates in the continuous space of discrete psychological labels of the first and second sets. For example, training module 108 may perform the comparison to determine an error, and then may perform techniques such as gradient descent and/or back propagation to train the regression model.

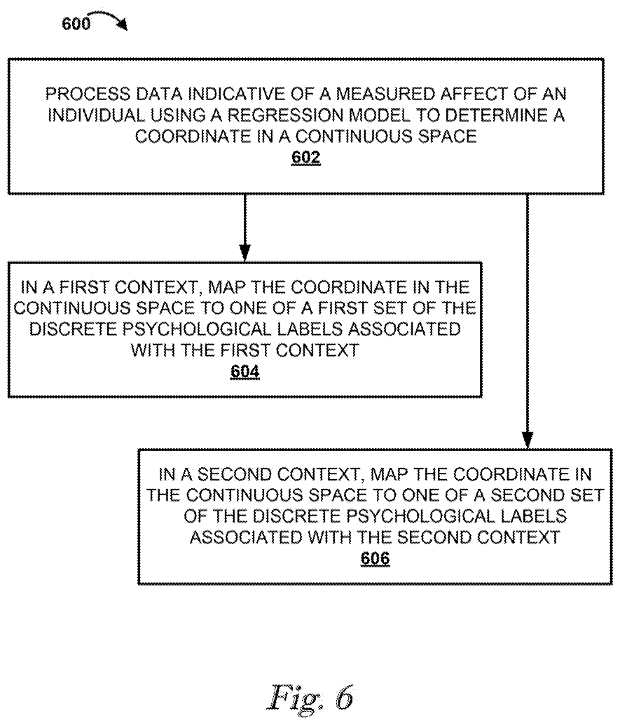

FIG. 6 depicts an example method for inferring psychological states, in accordance with various examples. For convenience, the operations of method 600 will be described as being performed by a system, which may include, for instance, psychological prediction system 100. The operations of method 600 may be reordered, and various operations may be added and/or omitted.

At block 602, the system, e.g., by way of inference module 104, may process data indicative of a measured affect of an individual using a regression model (e.g., MLP regressor model 456 and/or 466) to determine a coordinate in a continuous space. The continuous space may be indexed based on a plurality of discrete psychological labels, as depicted in FIGS. 2A-C, for instance.

In a first context, at block 604, the system, e.g., by way of inference module 104, may map the coordinate in the continuous space to one of a first set of the discrete psychological labels associated with the first context. In some examples, the system, e.g., by way of UI module 110, may then cause a computing device operated by a second individual to render output conveying that the first individual (i.e., the individual under consideration) exhibits the one of the first set of discrete psychological labels. For example, an English speaker may receive a psychological inference from an English-language set of discrete psychological labels aligned for the western cultural context.

In a second context, at block 606, the system may map the coordinate in the continuous space to one of a second set of the discrete psychological labels associated with the second context. In some examples, the system, e.g., by way of UI module 110, may then cause a second computing device operated by a third individual to render output conveying that the first individual exhibits the one of the second set of discrete psychological labels. For example, a Japanese speaker may receive an inference from a Japanese set of discrete psychological labels aligned for the Japanese cultural context.

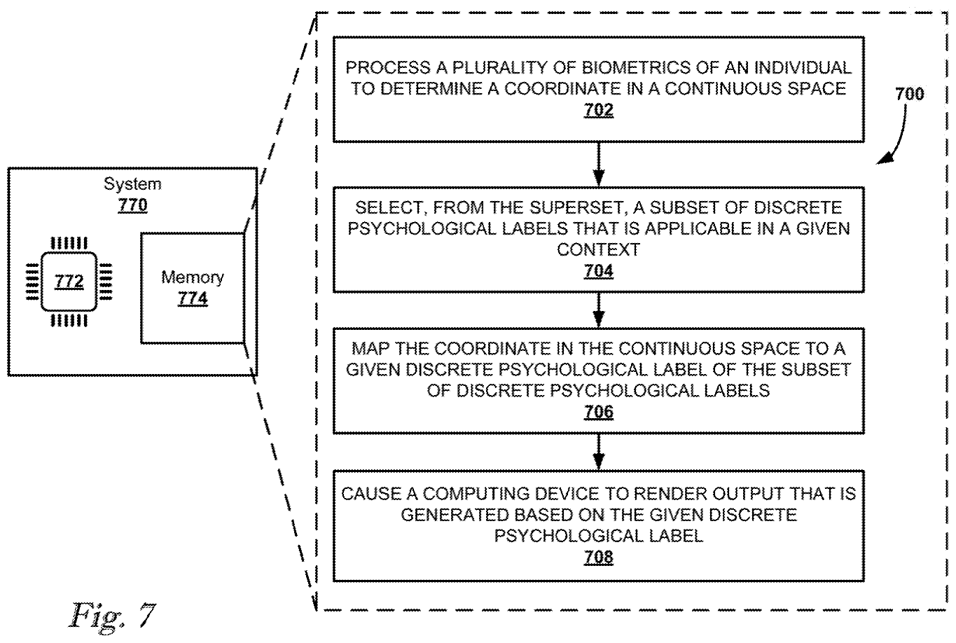

FIG. 7 shows a schematic representation of a system 770, according to an example of the present disclosure. System 770 includes a processor 772 and memory 774 that stores non-transitory computer-readable instructions 700 for performing aspects of the present disclosure, according to an example.

Instructions 702 cause processor 772 to process a plurality of biometrics of an individual (e.g., sensor-captured features of a facial expression, bodily movement/posture, voice, etc.) to determine a coordinate in a continuous space. In various examples, a superset of discrete psychological labels is mapped onto the continuous space.

Instructions 704 cause processor 772 to select, from the superset, a subset (e.g., a palette) of discrete psychological labels that is applicable in a given context. For example, if generating a psychological inference for a user in Brazil, a subset of discrete psychological labels generated from a Brazilian affectual dataset may be selected. If generating a psychological inference for a user in France, a subset of discrete psychological labels generated from a French affectual dataset may be selected. And so on. The quantity, size, and/or location of the regions representing the discrete psychological labels may vary as appropriate for, e.g., the cultural context of the user.

Instructions 706 cause processor 772 to map the coordinate in the continuous space to a given discrete psychological label of the subset of discrete psychological labels, e.g., using a Voronoi plot as described previously. Instructions 708 cause processor 772 to cause a computing device (e.g., personal computing device 114) to render output that is generated based on the given discrete psychological label. For example, UI module 110 may generate an HTML/XML document that is used by a personal computing device 114 to render a GUI based on the HTML/XML.

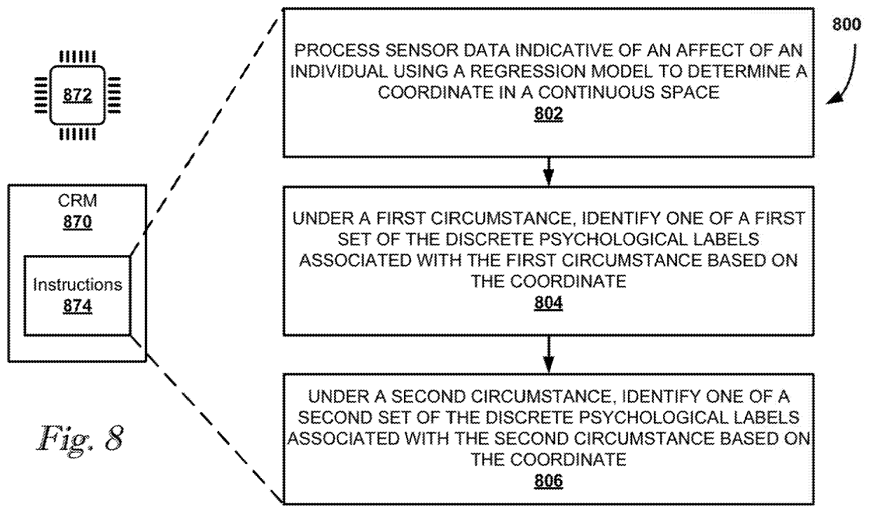

FIG. 8 shows a schematic representation of a non-transitory computer-readable medium (CRM) 872, according to an example of the present disclosure. CRM 870 stores computer-readable instructions 874 that cause the method 800 to be carried out by a processor 872.

At block 802, processor 872 may process sensor data indicative of an affect of an individual using a regression model to determine a coordinate in a continuous space. In various examples, a plurality of discrete psychological labels are mapped to the continuous space.

At block 804, processor 872 may, under a first circumstance, identify one of a first set of the discrete psychological labels associated with the first circumstance based on the coordinate. At block 806, processor 872 may, under a second circumstance, identify one of a second set of the discrete psychological labels associated with the second circumstance based on the coordinate.

Although described specifically throughout the entirety of the instant disclosure, representative examples of the present disclosure have utility over a wide range of applications, and the above discussion is not intended and should not be construed to be limiting, but is offered as an illustrative discussion of aspects of the disclosure.

What has been described and illustrated herein is an example of the disclosure along with some of its variations. The terms, descriptions and figures used herein are set forth by way of illustration and are not meant as limitations. Many variations are possible within the spirit and scope of the disclosure, which is intended to be defined by the following claims—and their equivalents—in which all terms are meant in their broadest reasonable sense unless otherwise indicated.

What is claimed is:

1. A method implemented using a processor, comprising:
processing data indicative of a measured affect of an individual using a regression model to determine a coordinate in a continuous space, wherein the continuous space is indexed based on a plurality of discrete psychological labels;
in a first context, mapping the coordinate in the continuous space to one of a first set of the plurality of discrete psychological labels associated with the first context; and
in a second context, mapping the coordinate in the continuous space to one of a second set of the plurality of discrete psychological labels associated with the second context.

2. The method of claim 1, wherein the individual is a first participant of a video conference, and the method comprises:
determining the first context based on a first signal associated with a second participant of a video conference; and
determining the second context based on a second signal associated with a third participant of the video conference.

3. The method of claim 2, further comprising:
causing a first computing device operated by the second participant to render output conveying that the individual exhibits the one of the first set of the plurality of discrete psychological labels; and
causing a second computing device operated by the third participant to render output conveying that the individual exhibits the one of the second set of the plurality of discrete psychological labels.

4. The method of claim 1, wherein the data indicative of the affect comprises an embedding generated based on a plurality of biometrics of the individual.

5. The method of claim 4, wherein the affect comprises multiple of:
a facial expression of the individual;
a characteristic of a posture of the individual; or
a characteristic of the individual's voice.

6. The method of claim 1, wherein mapping the coordinate in the continuous space to one of the first set of the plurality of discrete psychological labels is performed using a Voronoi plot that partitions the continuous space into regions close to each of the first set of the plurality of discrete psychological labels.

7. The method of claim 1, wherein the first set of the plurality of discrete psychological labels are in a first language and the second set of the plurality of discrete psychological labels are in a second language that is different than the first language.

8. The method of claim 1, wherein the continuous space comprises a two-dimensional space with a first axis corresponding to valence and a second axis corresponding to arousal.

9. A system comprising a processor and memory storing instructions that, in response to execution of the instructions by the processor, cause the processor to:
process a plurality of biometrics of an individual to determine a coordinate in a continuous space, wherein a superset of discrete psychological labels is mapped onto the continuous space;
select, from the superset of discrete psychological labels, a subset of discrete psychological labels that is applicable in a given context;
map the coordinate in the continuous space to a given discrete psychological label of the subset of discrete psychological labels; and
cause a computing device to render output that is generated based on the given discrete psychological label.

10. The system of claim 9, comprising instructions to preprocess the plurality of biometrics to generate an embedding, wherein the coordinate is determined based on application of the embedding across a regression model.

11. The system of claim 9, wherein the given context is determined based on a current activity of the individual.

12. The system of claim 9, wherein the continuous space comprises a two-dimensional space with a hedonic axis and an activation axis.

13. A non-transitory computer-readable medium comprising instructions that, in response to execution of the instructions by a processor, cause the processor to:
process sensor data indicative of an affect of an individual using a regression model to determine a coordinate in a continuous space, wherein a plurality of discrete psychological labels are mapped to the continuous space;
under a first circumstance, identify one of a first set of the plurality of discrete psychological labels associated with the first circumstance based on the coordinate; and
under a second circumstance, identify one of a second set of the plurality of discrete psychological labels associated with the second circumstance based on the coordinate.

14. The non-transitory computer-readable medium of claim 13, wherein the first circumstance comprises the first set of the discrete psychological labels being active based on user operation of an input device.

15. The non-transitory computer-readable medium of claim 13, wherein the first set of the plurality of discrete psychological labels comprises a first set of emotions that are expected to be observed under the first circumstance, and the second set of the plurality of discrete psychological labels comprises a second set of emotions that is incongruent with the first set of motions, and that are expected to be observed under the second circumstance.
